(19) United States
(12) Patent Application Publication
(10) Pub. No.: US 2005/0256184 A1
(43) Pub. Date: Nov. 17, 2005

(54) 1,2,4-Trioxanes and 1,2,4-trioxepanes

(76) Inventors: Paul M. O'Neill, Wavertree (GB); Richard Amewu, Liverpool (GB); Amira Mukhtar, Preston (GB); Stephen A. Ward, (US)

Correspondence Address: PENDORF & CUTLIFF, 5111 Memorial Highway, Tampa, FL, 33634-7356, US

(21) Appl. No.: 11/103,076

(22) Filed: Apr. 11, 2005

Related U.S. Application Data
(60) Provisional application No. 60/561,589, filed on Apr. 12, 2004.

(51) Int. Cl. A61K 31/335; C07D 323/04

(52) U.S. Cl. 514/452; 549/367

(57) ABSTRACT

Novel substituted 1,2,4-trioxanes and 1,2,4-trioxepanes useful as anti-malarial and/or anticancer agents, and an improved method for their preparation, preferably involving a thiol-olefin co-oxygenation (TOCO) reaction between an allylic alcohol and a ketone.

This utility application claims priority from Provisional Patent Application No. 60/561,589 filed under 37 C.F.R. § 1.53(c) on Apr. 12, 2004.

### BACKGROUND OF THE INVENTION

1. Field of the Invention

The present invention relates to novel substituted 1,2,4-trioxanes and 1,2,4-trioxepanes useful as anti-malarial and/or anticancer agents, and to an improved method for their preparation.

2. Discussion of Related Art

The 1,2,4-trioxane pharmcophore (structure 1) is an important functional group in medicinal chemistry. It is found in the artemisinin class of antimalarials such as artemether (structure 2) and artesunate, in which its reaction with heme (or free Fe(II)) generates cytotoxic radicals which cause parasite death. More recently, artemisinin derived 1,2,4-trioxane monomers and dimers have been shown to be potent inhibitors of cancer cell proliferation. A disadvantage with the semi-synthetic compounds described is that their production requires artemisinin as starting material. Artemisinin is extracted from the plant Artemisinia annua in low yield, a fact that necessitates significant crop-production. Therefore, there is much need for the development of new and improved approaches to the 1,2,4-trioxane functionality. Current literature methods for the synthesis of the 1,2,4-trioxane unit include the reaction of dioxetanes with carbonyls in the presence of Lewis acids, acid-catalysed cyclisation of hydroxyperoxyacetals with olefins and reactions of α-peroxy aldehydes with carbonyl compounds. All these routes provide the trioxane in moderate to low overall yields.

The numbering system of the basic 1,2,4-trioxane ring system (structure 1), and the antimalarial 1,2,4-trioxane artemether (structure 2) is shown in FIG. 1.

### SUMMARY OF THE INVENTION

Broadly, in a first aspect, the present invention is to provide novel substituted 1,2,4-trioxanes useful as anti-malarial and/or anticancer agents, and having the structure (A): 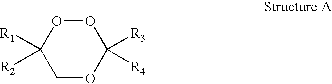
R¹=—CH₂OH, CHO, alkenyl, methyl sulfonyl aryl, methyl sulfinyl, methyl piperezinyl
R²=Aryl, alkyl
R³=Alkyl, cycloalky
R⁴=Alkyl, cycloalkyl
   or R³ and R⁴ together comprise cycloalkyl, including enantiomers, salts, and hydrates thereof.

In a second aspect, the invention provides a method for the synthesis of substituted 1,2,4-trioxanes of structure (A) according to a thiol-olefin co-oxygenation (TOCO) reaction between an allylic alcohol and a ketone.

In a third aspect, the invention concerns the extension of the methodology to the synthesis of functionalized spiro 1,2,4-trioxepanes of structure (B) by a simple one-pot procedure: 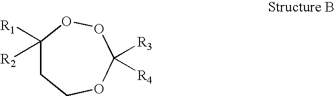
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
   wherein R³ and R⁴ may be cyclic ring systems such as adamantyl, cyclopentyl, cyclohexyl and cyclododecanyl, R¹=alkyl and R²=aryl.

In a fourth aspect, the invention provides a pharmaceutical formulation comprising as an active ingredient the 1,2,4-trioxane of structure (A) in a pharmaceutical carrier, and useful in the treatment of malaria and/or cancer.

In a fifth aspect, the invention provides a pharmaceutical formulation comprising as an active ingredient a 1,2,4-trioxepane of structure (B) in a pharmaceutical carrier, and useful in the treatment of malaria and/or cancer.

### BRIEF DESCRIPTION OF THE DRAWINGS

Figures are provided for ease of understanding of the invention, wherein:

FIG. 1 shows the numbering system for the basic 1,2,4-trioxane ring system (structure 1), and the antimalarial 1,2,4-trioxane artemether (structure 2), and

FIG. 2 shows X-ray structures of sulfide trioxane 4d and sulfone trioxane 8f.

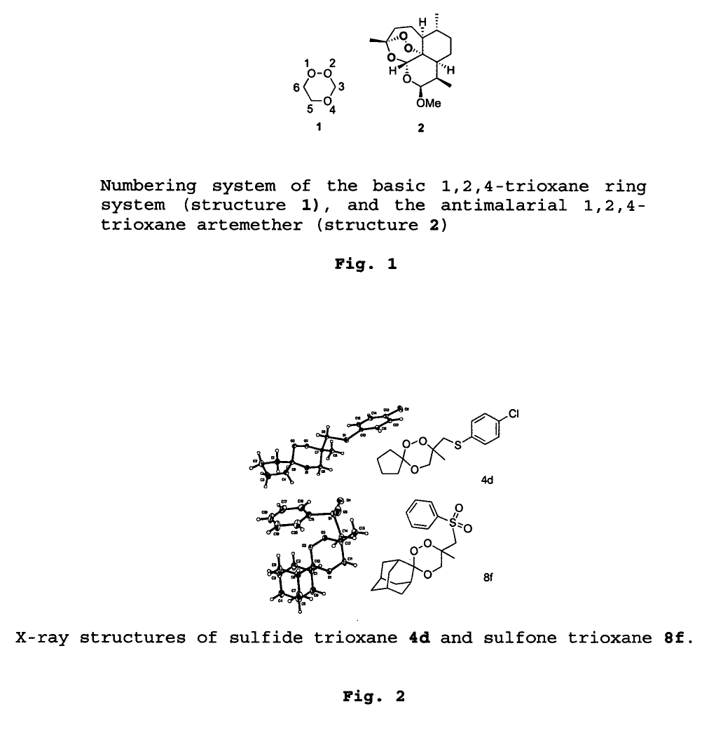

### DETAILED DESCRIPTION OF THE INVENTION

The following detailed examples have been provided merely to illustrate the invention and should not be construed as limitations on the inventive concept.

The mode of administration of the compounds of structure (A) and (B) or pharmaceutical formulations thereof can be oral, intra-muscular, subcutaneous or intravenous.

Pharmaceutical formulations containing the compound of structure (A) or (B) as active agent for treatment of malaria can be used in combination with other antimalarial compounds such as quinoline (amodiaquine) or quinoline menthanol (mefloquine). Pharmaceutical formulations containing the antimalarial endoperoxides of the present invention can be used in combination with other antimalarial compounds such as halofantrine, benflumetol and LAPDAP.

Suitable salts of the compounds according to structure (A) or (B) include acid addition salts and these may be formed by reaction of a suitable compound of structure (A) or (B) with a suitable acid, such as an organic acid or a mineral acid.

Any alkyl or alkenyl group, unless otherwise specified, may be linear or branched and may contain up to 20 carbon atoms. Preferred alkyl moieties are methyl and ethyl.

An aryl group may be any monocyclic or polycyclic aromatic hydrocarbon group and may contain from 6 to 24, preferably 6 to 14, carbon atoms. Preferred aryl groups include phenyl, and naphthyl groups.

A cycloalkyl group may be any saturated cyclic hydrocarbon group and may contain from 3 to 30 carbon atoms. Preferred cycloalkyl groups are adamantyl, cyclopentyl, cyclohexyl, and cyclododecanyl groups. Cycloalkyl groups may thus include polycyclic cyclic alkyls, which contain more than one ring system. Such ring systems may be “fused”, that is, adjacent rings have two adjacent carbon atoms in common, “bridged”, that is, the rings are defined by at least two common carbon atoms (bridgeheads) and at least three acyclic chains (bridges) connecting the common carbon atoms, or “spiro” compounds, that is, adjacent rings are linked by a single common carbon atom.

When any of the foregoing substituents are designated as being optionally substituted, the substituent groups which are optionally present may be any one or more of those customarily employed in the development of pharmaceutical compounds and/or the modification of such compounds to influence their structure/activity, stability, bioavailability or other property. Specific examples of such substituents include, for example, halogen atoms, nitro, cyano, hydroxyl, cycloalkyl, alkyl, alkenyl, haloalkyl, cycloalkyloxy, alkoxy, haloalkoxy, amino, alkylamino, dialkylamino, formyl, alkoxycarbonyl, carboxyl, alkanoyl, alkylthio, alkylsulphinyl, alkylsulphonyl, alkylsulphonato, arylsulphinyl, arylsulphonyl, arylsulphonato, carbamoyl, alkylamido, aryl, aralkyl, optionally substituted aryl, heterocyclic and alkyl- or aryl-substituted heterocyclic groups. A halogen atom may be a fluorine, chlorine, bromine or iodine atom and any group which contains a halo moiety, such as a haloalkyl group, may thus contain any one or more of these halogen atoms.

The preferred methods of synthesis of the compounds of structure (A) and (B) are now illustrated in the following non-limiting examples.

As discussed above, a second aspect the invention relates to the synthesis of new antimalarial endoperoxides utilizing a thiol-olefin co-oxygenation (TOCO) reaction to generate bicyclic peroxides structurally related to the yingzhaosu A (Scheme 1, reaction 1). In a third embodiment of the invention, by replacement of a terpene with an allylic alcohol, this methodology can be extended to a new synthesis of functionalised Spiro 1,2,4-trioxanes by a simple one pot procedure (Scheme 1, reaction 2).

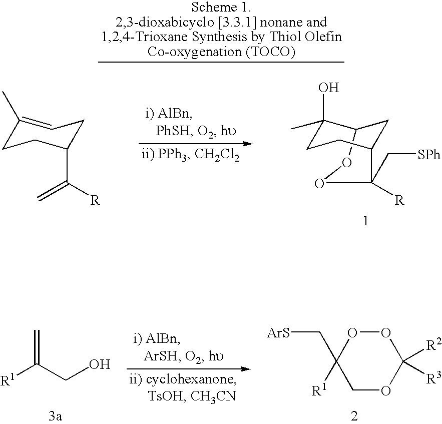

Scheme 2 illustrates the mechanism for the TOCO/condensation reaction. Phenylthiyl radical, generated from phenylthiol through initiation with AIBN/hv, attacks the double bond of the allyl alcohol 3a in a Markovnikov fashion to generate a tertiary carbon radical 5a. This radical traps oxygen to form a peroxy radical 6a. Radical hydrogen abstraction from thiophenol produces the α-hydroxyperoxide 7a and regenerates phenylthiyl radical to propagate the reaction. The α-hydroxyperoxide was subsequently shown to undergo smooth condensation with cyclohexanone in the presence of a catalytic amount of tosic acid, to generate the 1,2,4-trioxane 4a.

FIG. 2 depicts X-ray crystal structures for the trioxane 4d and the sulfone generated from 4f.

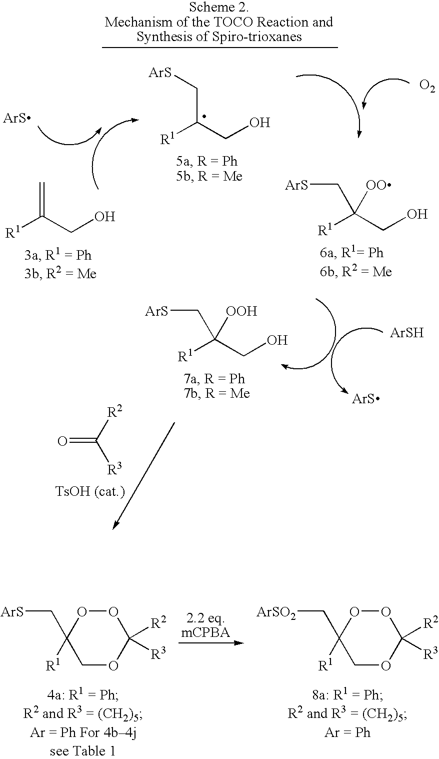

Application of the method described in Scheme 2, to various combinations of ketones and allylic alcohols, afforded the series of spiro-trioxanes shown in Table 1. Considering the complex sequence of events that must occur to obtain the α-hydroxyperoxide intermediate and subsequent carbonyl condensation, the overall sequence proceeds in good yield. By-products generated from oxidation of thiophenol are observed in very low yields since, as described above, the reaction is carried out under high dilution conditions to minimize the potential of peroxy radical 6 to engage in side reactions

FIG. 2 shows the X-ray structures of sulfide trioxane 4d and sulfone trioxane 8f.

Sulfide trioxanes (4e, 4f and 4j) and their corresponding sulfones (8e, 8f and 8j) were tested for antiparasitic activity versus chloroquine resistant Plasmodium falciparum. The preferred method for the synthesis of sulfone trioxanes involves the reaction of the appropriate sulfide with 2.2 equivalents of mCPBA. Structures of novel sulfones are depicted in Table 2. All of the analogues display moderate antimalarial activity, with the exception of trioxane 8j which exhibits potent activity (72 nM) (Table 3). This compound is currently being examined for its in vivo antimalarial activity versus Plasmodium berghei.

In addition to providing facile access to the trioxane pharmacophore, the TOCO/carbonyl condensation protocol generates a methylthiophenyl group in the resulting trioxane. This functionality has proven useful for further manipulation of the structure to generate chemically diverse groups. Scheme 3 illustrates the synthesis of a formyl-substituted trioxane (9b) via thiol oxidation using stoichiometric mCPBA followed by exposure of the sulfoxide 9a to Pummerer conditions. The resultant carbonyl group in 9b readily undergoes numerous condensation and nucleophilic substitution reactions, imparting a high degree of structural flexibility to a pharmacophore which is of great interest in current medicinal chemistry.

TABLE 1
Trioxanes synthesized via intermolecular
trapping of the hydroxperoxide products 7a and 7b of the TOCO
reaction with cyclic ketones.

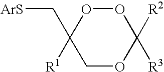

| Allylic | | | |
|---|---|---|---|
| alcohol | Ketone | Trioxane | Yield % |
| 3a | cyclohexanone | 4a: R¹ = Ph; R² | 68 |
| | | and R³ = (CH₂)₅; | |
| | | Ar =Ph | |
| 3a | cyclopentanone | 4b: R¹ = Ph; R² | 54 |
| | | and R³ = (CH₂)₄; | |
| | | Ar = Ph | |
| 3b | cyclohexanone | 4c: R¹ = Ph; R² | 53 |
| | | and R³ = (CH₂)₅; | |
| | | Ar=Ph | |
| 3b | cyclopentanone | 4d: R¹ = Ph; R² | 40 |
| | | and R³ = (CH₂)₅; | |
| | | Ar = p-Cl-Ph | |
| 3b | cyclobutanone | 4e: R¹ = Ph; R² | 61 |
| | | and R³ = (CH₂)₃; | |
| | | Ar = Ph | |
| 3b | adamantanone | 4f: R¹ = Me; | 42 |
| | | R²CR³ = | |
| | | adamantylidene; | |
| | | Ar = Ph | |
| 3b | 4-t-Bu- | 4g: R¹ = Me; | 80 |
| | cyclohexanone | R²CR³ = 4-t-Bu- | |
| | | cyclohexyllidene; | |
| | | Ar = Ph | |
| 3b | 1,4- | 4h: R¹ = Me; | 25 |
| | cyclohexadione | R²CR³ = 4- | |
| | | oxocyclohexylidene; | |
| | | Ar = Ph | |
| 3b | cyclohexanone | 4i: R¹ = Me; R² | 78 |
| | | and R³ = (CH₂)₆; | |
| | | Ar = p-Cl-Ph | |
| 3b | cyclododecanone | 4j: R¹ = Me; R² | 68 |
| | | and R³ = (CH₂)₁₁; | |
| | | Ar = Ph | |
| 3b | cyclododecanone | 4k: R¹ = Me; R² | 73 |
| | | and R³ = (CH₂)₁₁; | |
| | | Ar = p-Cl-Ph | |
| 3b | cyclododecanone | 4l: R¹ = Me; R² | 64 |
| | | and R³ = (CH₂)₁₁; | |
| | | Ar = 2-Naphthyl | |
| 3b | adamantanone | 4m: R¹ = Me; | 75 |
| | | R²CR₃ = | |
| | | adamantylidene; | |
| | | Ar = p-Cl- Ph | |
| 3b | tetrahydro- pentalene- 2,5(1H,3H)- dione | 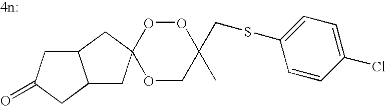 | 65 |

TABLE 2
Structures of Sulfonyl Trioxanes

| | |
|---|---|
| 8c | 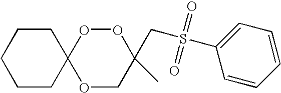 |
| 8d | 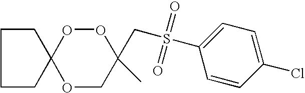 |
| 8e | 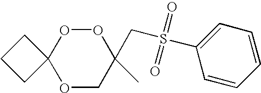 |
| 8f | 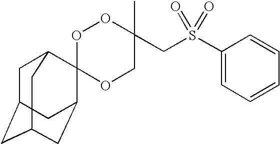 |
| 8g | 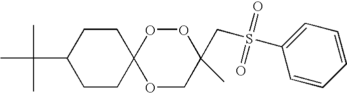 |
| 8h | 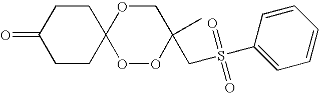 |
| 8i | 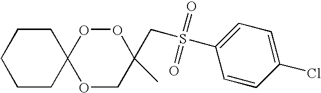 |
| 8k | 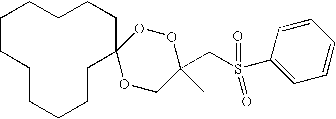 |
| 8m | 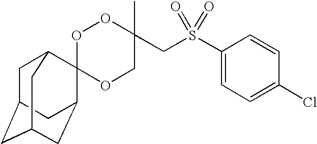 |

Table 3. In Vitro Antimalarial Activity of Spiro Trioxanes versus K1 Plasmodium falciparum*

| Trioxane | IC50 (nM) | SD ± Mean |
|---|---|---|
| 4e | 314 | 68 |
| 4f | 285 | 54 |
| 4j | 135 | 53 |
| 8f | 329 | 61 |
| 8i | 35 | 17 |
| 8j | 72 | 42 |
| Artemisinin | 12 | 80 |
| Chloroquine | 210 | 55 |

8f and 8j are the sulfones derived from 4f and 4j respectively

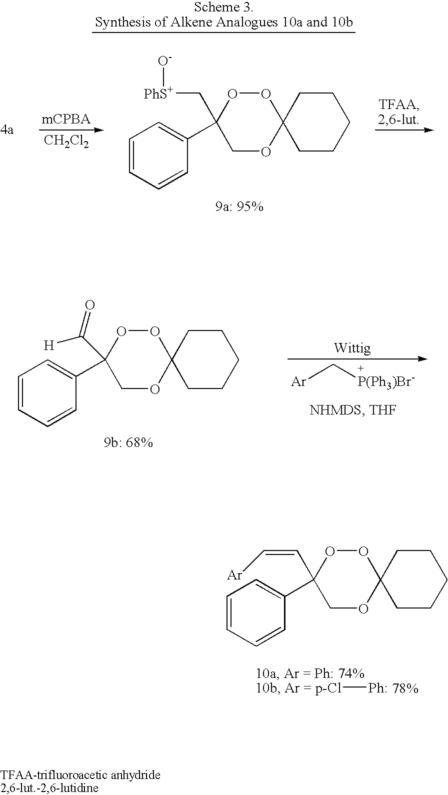

For example, Wittig reactions on 9b provides vinyl substituted trioxane analogues 10a and 10b in excellent yields.

Aldehydes such as 9b are also convenient precursors for the synthesis of desirable piperazinyl functionalised 1,2,4-trioxanes. For example, using adamantyl functionalised 4f, sulfoxidation and a modified Pummerer reaction provides the aldehyde 11. Reductive amination of 11 with N-phenyl piperazine provides the substituted 1,2,4-trioxane 12 that can be formulated as a water soluble salt. This procedure in essence is unlimited and any amine can be incorporated using the reductive amination approach depicted in Scheme 4.

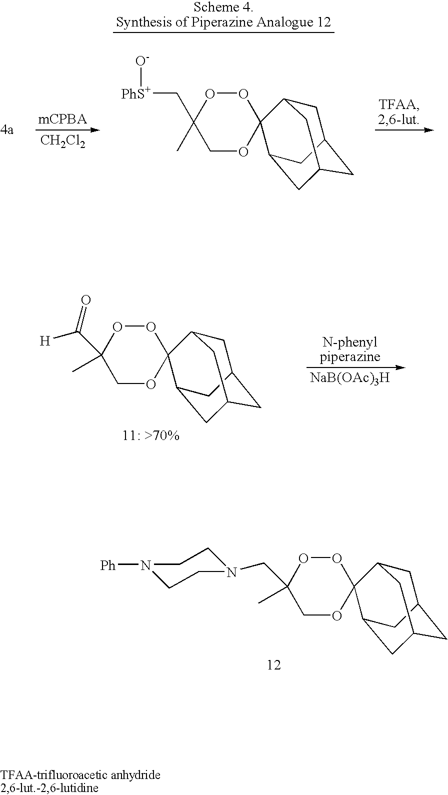

Using analogues of the aldehyde 9b and 11 we have also have prepared several vinyl substituted ester derivatives by simple Wittig protocols. Representative examples are 13a-13f.

TABLE 4

| | |
|---|---|
| 13a | 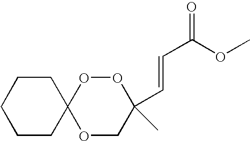 |
| 13b | 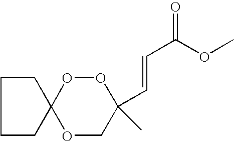 |
| 13c | 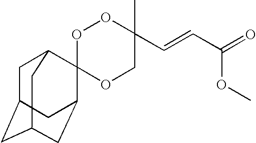 |
| 13d | 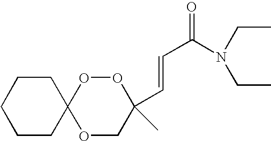 |
| 13e | 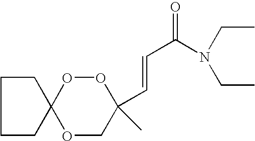 |
| 13f | 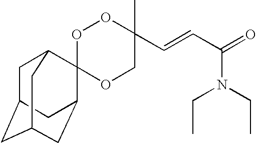 |

The methodology described here provides virtually unlimited access to the generic 1,2,4-trioxane structure A and the preferred substituents defined as follows: 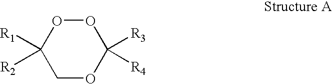
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl

Particularly preferred structures are represented by structure A, where R³ and R⁴ are cyclic ring systems such as adamantyl, cyclopentyl, cyclohexyl and cyclododecanyl, R¹=alkyl and R²=aryl.

1,2,4-Trioxepanes

By applying the TOCO reaction to homoallylic alcohols, this methodology enables access to the corresponding 1,2,4-trioxepane pharmacophore. Scheme 5 depicts the process for the synthesis of representative examples 14a-14c. Oxidation of these sulfides using mCPBA provides the corresponding sulfones 15a-15c. A summary of the structures of trioxepanes obtained is shown in Table 5.

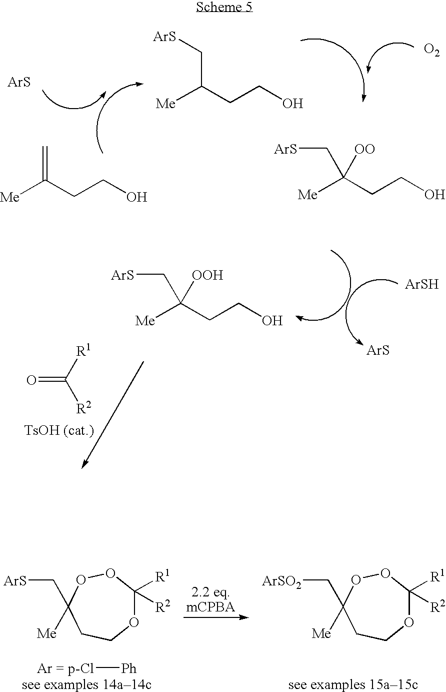

TABLE 5

| | |
|---|---|
| 14a | 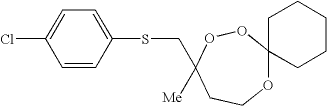 |
| 14b | 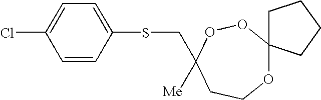 |
| 14c | 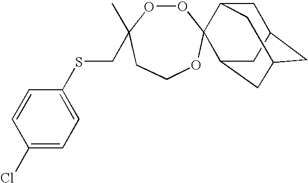 |
| 15a | 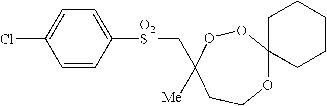 |
| 15b | 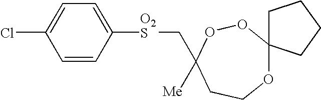 |
| 15c | 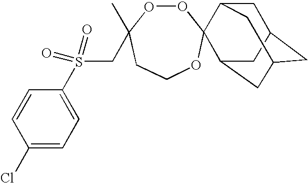 |

Scheme 6 describes the synthesis of vinyl ester derivatives 16a-16c by a three step sequence reported earlier in the 1,2,4-trioxane series.

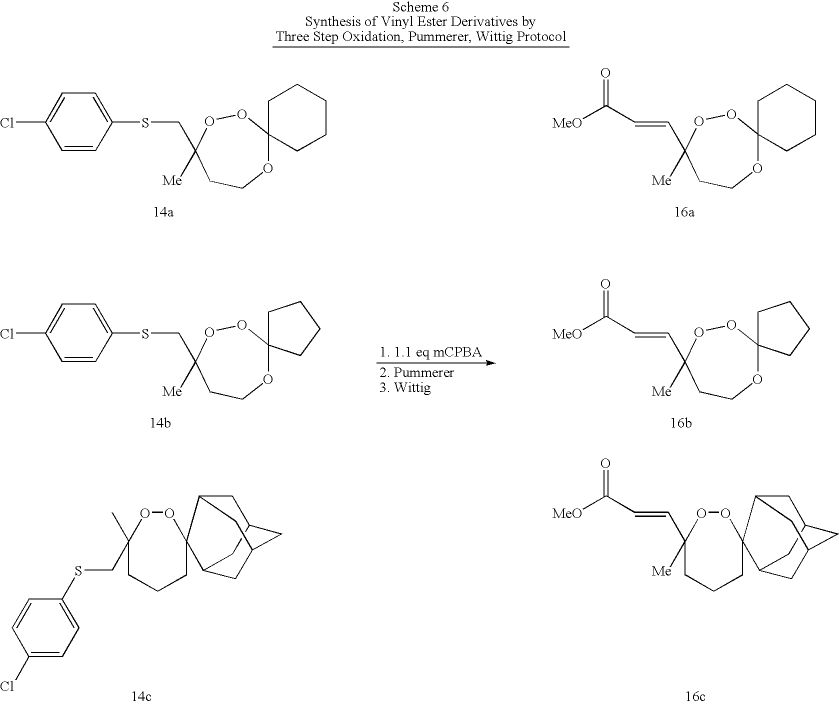

The methodology described here provides virtually unlimited access to the generic 1,2,4-trioxepane structure B and the preferred substituents.

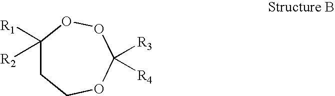

R¹=—CH₂OH, CHO, alkenyl, methyl sulfonyl aryl, methyl sulfinyl, methyl piperazinyl
R²=Aryl, alkyl
R³=Alkyl, cyclo alkyl
R⁴=Alkyl, cycloalkyl

Particularly preferred structures are represented by Structure (B), where R³ and R⁴ are cyclic ring systems such as adamantyl, cyclopentyl, cyclohexyl and cyclododecanyl, R¹=alkyl and R²=aryl.

General Procedure (1) for the TOCO Synthesis of 1,2,4-Trioxanes.

8-(4-Chloro-phenylsulfanylmethyl)-8-methyl-6,7,10 trioxa-spiro[4.5]decane (4d).

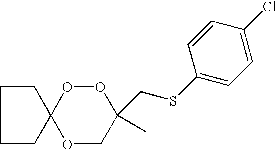

A 2-necked 250 ml round bottomed flask was charged with a solution of 2-methyl-2-propen-1-ol (200 mg, 0.23 ml, 2.77 mmol) and AIBN (31 mg, 1.89 mmol) in acetonitrile (46 ml). The reaction vessel was flushed with oxygen for several minutes at 0° C. then stoppered and kept under a positive pressure of pure oxygen, with the aid of two big oxygen balloons. The reaction mixture was vigorously stirred and UV irradiated (at 0° C.) using an externally mounted 100W BLAK-RAY UV lamp at a distance of 5-7 cm, with the simultaneous addition of 4-chlorothiophenol (500 mg, 3.46 mol 1.25 equiv) solution in acetonitrile (13 ml) over a period of 30 mins. After complete addition, the reaction was left to continue stirring at 0° C., for 4-6 hours or until consumption of starting materials (monitored by tlc). The reaction vessel was then cooled to −10° C., flushed with nitrogen and a solution of the cyclopentanone (780 mg, 0.82 ml, 6.94 mmol), in dichloromethane (13 ml) was added, followed by catalytic amounts of tosic acid. The mixture was left stirring at −10° C., and allowed to cool slowly to room temperature overnight. Removal of the solvent in vacuo and purification by column chromatography yielded the desired endoperoxide (4d) as a crystalline white solid (340 mg, 40%). Mp 69.8-70° C.; IR (neat) 2355, 1644, 1477, 1331, 1300, 1189, 1127, 1092, 1042, 1006, 966, 847, 815 and 722 cm⁻¹; ¹H NMR (400 MHz, CDCl₃) δ_{H} 7.34 (d J=8.59 2H aromatic signal), 7.25 (dd, J=8.58, 1.52, 2H aromatic signal) 3.79 (d, J=11.6, 1H, —CH trioxane moiety), 3.68 (d, J=11.3, 1H, —CH trioxane moiety), 3.54 (bs, 2H, —CH₂), 1.75 (bm, 8H, —CH₂CH₂CH₂CH₂—), 1.12 (bs, 3H, —CH₃); ¹³CNMR (100 MHz, CDCl₃) 132.46, 130.94, 129.39, 114.62, 79.27, 66.55, 38.49, 37.52, 32.29, 25.01 and 20.44; MS (ES+) m/z 337.2 [M+Na]⁺ (100), 353.1 [M+K]⁺ (31), 651.3 [2M+Na]⁺ (14); HRMS m/z calcd for C₁₅H₁₉O₃SClNa [M⁺+Na] 337.0628 found, 337.0641; Elemental analysis C, 57.37; H, 6.08; (required values; C, 57.23; H, 6.08).

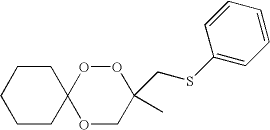

3-Methyl-3-phenylsulfanylmethyl-1,2,5-trioxa-spiro[5.5]undecane (4c). This compound was synthesised in 53% yield according to General Procedure 1 using thiophenol and cyclohexanone. Mp 45-46° C.; IR (nujol) 3020 (weak, aryl-H), 1711 (CO—O peroxide bond), 1585 (medium, aromatic ring), 1481, 1311, 1158, 1095, 945, 735 and 689 cm⁻¹; ¹H NMR (400 MHz, CDCl₃) δH 7.48 (d, J=7.60, 2H, aromatic signal), 7.34 (t, J=7.32, 1H aromatic signal), 7.27 (t, J=7.48, 2H, aromatic), 3.77 (bs, 2H, —CH₂), 2.99 (d, J=3.16, 1H, O—CH), 2.95 (d, J=3.20, 1H, O—CH), 1.55 (bm, 10H, —C₆H₁₀), 0.88 (s, 3H, CH₃); MS (ES+) m/z 317.2 [M+Na]⁺ (33), HRMS m/z calcd for C₁₆H₂₂O₃SNa [M⁺+Na] 317.1187 found, 317.1179; Elemental analysis C, 65.99; H, 7.86; (required values; C, 65.31H, 7.54).

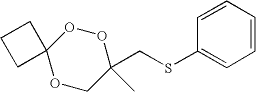

7-Methyl-7-phenylsulfanylmethyl-5,6,9-trioxa-spiro[3.5]nonane (4e). This compound was synthesised in 61% yield according to General Procedure 1 using thiophenol and cyclobutanone. ¹H NMR (400 MHz, CDCl₃) δH 7.34 (m, 5H, aromatic signal), 3.75 (d, J=11.76, 1H, O—CH), 3.55 (d, J=11.31, 1H, O—CH), 3.10 (bs, 2H, —CH₂), 2.20 (bm, 4H, cyclobutanone moiety), 1.76 (bm, 2H, cyclobutanone moiety) 1.31 (s, 3H, CH₃); 13C NMR (100 MHz, CDCl₃) δ_{c} 133.00, 129.30, 127.97, 126.55, 104.65, 79.73, 73.08, 41.03, 37.32, 20.28 and 11.10. MS (ES+) m/z 289.1 [M+Na]⁺ (89), 305.1 [M+K]⁺ (100); HRMS m/z calcd for C₁₄H₁₈O₃SNa [M⁺+Na] 289.0874 found, 289.0877. Elemental analysis C, 63.01; H, 6.55; (required values; C, 63.13; H, 6.81).

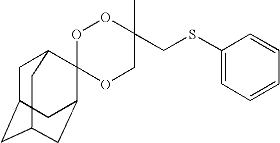

Adamantyl Trioxane (4f). This compound was prepared in 42% yield as an oil according to General Procedure 1 from thiophenol and admant-2-one; IR (neat) 3062 (weak aryl-H), 2913 (—CH₂, strong), 2860 (—CH weak), 1795 (O—O peroxide bond weak), 1598 (medium, phenyl ring) cm⁻¹; ¹H NMR (400 MHz, CDCl₃) δ_{H} 7.79 (d, J=3.64 2H aromatic signal), 7.41 (t, J=7.76, 1H, aromatic) 7.26 (dd, J=7.8, 3.36, 2H aromatic signal), 3.60 (bs, 2H, O—CH₂), 2.60 (bs, 2H, —CH₂), 1.75 (bm, 14H, adamantane moiety), 1.15 (s, 3H, —CH₃); ¹³CNMR (100 MHz, CDCl₃) 135.88, 128.77, 126.34, 104.69, 79.27, 73.02, 39.54, 37.55, 33.74, 33.17, 27.80 and 20.64; MS (ES+) m/z 369.15 [M+Na]⁺ (100), 370.15 [M+1+Na]⁺ (21), 385.13 [M+K]⁺ (12); HRMS m/z calcd for C₂₀H₂₆O₃SNa [M⁺+Na] 369.1489 found, 369.1500; Elemental analysis C, 69.87; H, 7.24; (required values; C, 69.33; H, 7.56).

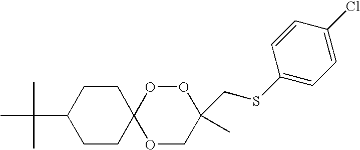

9-Tert-butyl-3-(4-chloro-phenylsufanylmethyl)-3-methyl-1,2,5-trioxa-spiro[5.5]undecane (4g).

This compound was prepared in 80% yield according to General Procedure 1 from thiophenol and 4-tert butyl cyclohexanone. ¹H NMR (400 MHz, CDCl₃) OH 7.34 (d, J=8.4, 2H aromatic), 7.25 (d, J=8.6, 2H aromatic), 3.75 (bs, 2H, O—CH₂), 3.50 (bs, 2H, —CH₂), 1.68 (bs, 4H, cyclohexyl peak), 1.36 (bm, 1H, cyclohexyl peak), 1.14, (bs, 3H, —CH₃), 1.05 (bm, 1H, cyclohexyl peak), 0.85 (s, 9H, ^{t}butyl peak); ¹³C NMR (100 MHz, CDCl₃) 138.87, 135.96, 132.33, 129.70, 102.72, 79.33, 64.16, 47.96, 38.70, 34.50, 32.71, 28.20, 23.37 and 20.60. MS (ES+) m/z 407.14 [M+Na]⁺ (100), 423.13 [M+K]⁺ (18); HRMS m/z calcd for C₂₀H₂₉O₃SClNa [M⁺+Na] 407.1424 found, 407.1442; Elemental analysis C, 63.50; H, 7.91; (required values; C, 62.38H, 7.59).

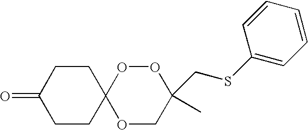

Trioxane Ketone (4h). This compound was prepared according to General Procedure in 25% yield from thiophenol and 1,4-cyclohexadione. MS (ES+) m/z 331 [M+Na]⁺ (100), 347 [M+K]⁺ (53), 363 [M+CH₃OH+Na]⁺ (78); HRMS m/z calcd for C₁₆H₂₀O₄SNa [M⁺+Na] 331.0980 found, 331.0985; Elemental analysis C, 62.05, H, 6.58; (required values; C, 62.31; H, 6.54).

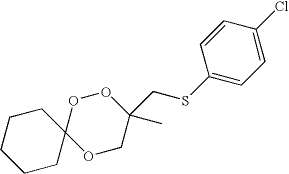

3-(4-Chloro-phenylsulfanylmethyl)-3-methyl-1,2,5-trioxa-spiro[5.5]undecane (4i). This compound was prepared in 78% yield according to general procedure 1 from p-chlorothiophenol and cyclohexanone. ¹H NMR (400 MHz, CDCl₃) δH 7.35 (d, J=8.59, 2H, aromatic), 7.24 (d, J=8.74, 2H aromatic), 3.75 (bd, 2H, O—CH₂), 3.48 (bs, 2H, —CH₂), 1.51 (bm, 10H, —C₆H₁₀), 1.20 (bs, 3H, CH₃); ¹³C NMR (100 MHz, CDCl₃) 139.78, 133.61, 131.83, 130.05, 103.29, 79.84, 73.50, 36.90, 30.88, 26.24, 22.72 and 20.70; MS (ES+) m/z 329.1 [M+H]⁺ (21), 351.1 [M+Na)⁺ (100), 367.1 [M+K]⁺ (20), 679.2 [2M+Na]⁺ (25); HRMS m/z calcd for C₁₆H₂₁O₃SClNa [M⁺+Na] 351.0798 found, 351.0805; Elemental analysis C, 59.40; H, 6.5; (required values; C: 58.44; H, 6.44).

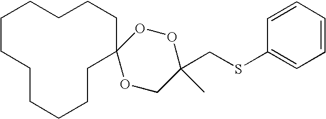

3-Methyl-3-phenylsulfanylmethyl-1,2,5-trioxa-spiro[5.11]heptadecane (4j).

This compound was prepared in 68% yield according to General Procedure 1 from thiophenol and cyclododecanone. Mp 89° C.; ¹H NMR (400 MHz, CDCl₃) OH 7.43 (d, J=7.64, 2H, aromatic), 7.26 (t, J=7.8, 2H, aromatic), 7.17 (t, J=7.48, 1H, aromatic), 3.75 (bs, 2H, —CH₂), 3.53 (bs, 2H, —O—CH₂), 1.48 (s, 3H, CH₃), 1.31 (bm, 22H, —C₁₂H₂₂); MS (ES+) m/z 401.1 [M+Na]⁺ (100), 417.1 [M+K]⁺ (24), 779.3 [2M+Na]⁺ (13); HRMS m/z calcd for C₂₂H₃₄O₃SNa [M⁺+Na] 401.2126 found, 401.2130; Elemental analysis C, 70.17; H, 9.27; (required values; C, 69.8; H, 9.05).

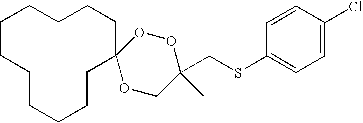

3-(4-Chloro-phenylsulfanylmethyl)-3-methyl-1,2,5-trioxa-spiro[5.11]heptadecane (4k). This compound was prepared in 73% yield according to General Procedure 1 from p-chlorothiophenol and cyclododecanone. Mp 93-94° C.; ¹H NMR (400 MHz, CDCl₃) δ_{H} 7.40 (d, J=8.40, 2H, aromatic), 7.35 (d, J=8.44, 2H, aromatic), 3.75 (bs, 2H, —O—CH₂), 3.49 (bs, 2H, —CH₂), 1.57 (s, 3H, CH₃), 1.35 (bm, 22H, —C₁₂H₂₂); ¹³C NMR (100 MHz, CDCl₃) 132.13, 130.56, 129.31, 128.99, 106.30, 78.77, 65.98, 26.04, 22.31, 22.27, 22.02, 21.74 and 20.26. MS (ES+) m/z 435.17 [M+Na]⁺ (74), 413 [M+H]⁺ (19), 847.36 [2M+Na]⁺ (100); HRMS m/z calcd for C₂₂H₃₃O₃SClNa [M⁺+Na] 435.1737 found, 435.1735; Elemental analysis C, 61.19; H, 6.84; (required values; C, 63.98; H, 8.05).

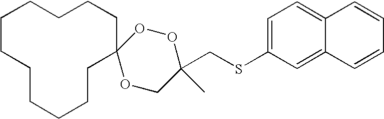

3-Methyl-3-(naphthalen-2-ylsulfanylmethyl)-1,2,5-trioxa-spiro[5.11]heptadecane (41). This compound was prepared in 64% yield according to General Procedure 1 from 2-napthalenethiol and cyclododecanone. ¹H NMR (400 MHz, CDCl₃) δ_{H} 7.97 (d, J=1.75, 1H, aromatic), 7.85 (d, J=1.43, 1H, aromatic), 7.76 (t, J=8.91, 1H, aromatic), 7.68 (d, J=8.58, 2H), 7.64 (d, J=8.43, 2H, aromatic), 4.62 (d, J=3.5, 2H, —CH₂), 4.14 (d, J=7.15, 1H, —O—CH), 4.10 (d, J=7.15, 1H, —O—CH), 1.72 (bm, 2H), 1.53 (s, 3H, CH₃) 1.28 (bm, 20H); ¹³C NMR (100 MHz, CDCl₃) 134.69, 133.98, 131.87, 130.05, 128.80, 127.86, 126.62, 113.65, 72.37, 68.18, 40.79, 33.88, 25.06, 24.69, 22.83 and 19.81. MS (ES+) m/z 451.30 [M+Na]⁺ (56), 468.20 [M+K]⁺ (10); HRMS m/z calcd for C₂₆H₃₆O₃SNa [M⁺+Na] 451.2283 found, 451.2296; Elemental analysis C, 73.91; H, 8.33; (required values; C, 72.86; H, 8.47).

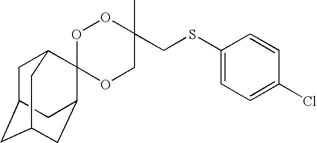

Adamantyl trioxane (4m). This compound was prepared in 75% yield according to General Procedure 1 from adamant-2-one and p-chlorothiophenol. ¹H NMR (400 MHz, CDCl₃) 7.34 (d, J=8.56, 2H, aromatic), 7.24 (d, J=8.60, 2H, aromatic), 3.74 (bs, 2H, —O—CH₂), 3.53 (bs, 2H, —CH₂), 1.76 (bm, 14H, adamantane moiety), 1.14 (s, 3H, CH₃); ¹³C NMR (100 MHz, CDCl₃) 132.79, 130.91, 129.70, 129.70, 129.61, 104.74, 79.14, 63.64, 37.50, 33.62, 31.35, 27.48 and 20.61; MS (ES+) m/z 403.1 [M+Na]⁺ (100), 419.1 [M+K]⁺ (19), 783.2 [2M+Na]⁺ (7); HRMS m/z calcd for C₂₀H₂₅O₃SClNa [M⁺+Na] 403.1111 found, 403.1120; Elemental analysis C, 62.77; H, 6.73; (required values; C, 63.06; H, 6.62).

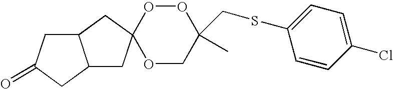

Trioxane (4n). This compound was prepared in 65% yield according to General Procedure 1 from tetrahydropentalene-2,5 (1H, 3H)-dione and p-chlorothiophenol.

¹H NMR (400 MHz, CDCl₃) 7.34 (d, J=8.44, 2H, aromatic), 7.26 (d, J=8.28, 2H, aromatic), 3.80 (d, J=11.76, 1H, O—CH), 3.66 (d, J=10.64, 1H, O—CH), 2.89 (bs, 2H, —CH₂), 2.5 (m, 4H), 2.15 (bm, 4H), 1.76 (bm, 2H); ¹³C NMR (100 MHz, CDCl₃) 133.59, 132.59, 131.03, 129.44, 128.92, 108.88, 79.53, 66.80, 44.51, 38.67, 37.79, 36.83 and 20.44. MS (ES+) m/z 391 (M+Na]⁺ (100), 407 [M+K]⁺ (17), HRMS m/z calcd for C₁₈H₂₁O₄SClNa [M⁺+Na) 391.0747 found, 391.0757; Elemental analysis C, 57.47; H, 5.54; (required values; C, 58.61; H, 5.74).

General Procedure 2 Synthesis of Sulfones

A solution of the sulfide starting material (0.3 mmol, 1 equiv) and mCPBA (0.75-0.9 mmol, 2.5-3.0 equiv) in 5 ml of CH₂Cl₂ was stirred for 4-6 h at rt. After consumption of the more polar intermediate sulfoxide (monitored by tlc), the mixture was poured into a saturated solution of cold 5% K₂CO₃ solution. The mixture was then extracted with DCM, the organic layer separated, dried over Na₂SO₄ and evaporated. Purification of the residue by column chromatography gave the desired sulphone compounds.

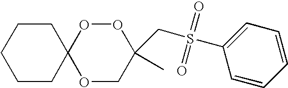

3-Benzenesulfonylmethyl-3-methyl-1,2,5-trioxa-spiro[5.5]undecane (8c). This compound was prepared in 75% yield according to General Procedure 2. Mp 88-89° C.; ¹H NMR (400 MHz, CDCl₃) δ_{H} 7.96 (d, J=7.00, 2H, aromatic signal), 7.65 (t, J=7.64, 1H aromatic signal), 7.57 (t, J=7.76, 2H, aromatic), 3.97 (bd, 2H, —CH₂), 3.80 (d, J=12.08, 1H, O—CH), 2.95 (bd, J=13.52, 1H, O—CH), 1.50 (bm, 10H, —C₆H₁₀), 1.38 (s, 3H, CH₃); ¹³C NMR (100 MHz, CDCl₃) 141.47, 133.98, 129.54, 128.23, 64.91, 43.08, 25.77, 22.58, 22.46 and 20.46. MS (ES+) m/z 349.2 [M+Na]⁺ (100), 365.2 [M+K]⁺ (7), 675.4 [2M+Na]⁺ (98); HRMS m/z calcd for C₁₆H₂₂O₅SNa [M+Na]⁺ 349.1086 found, 349.1072; Elemental analysis C, 59.00; H, 6.83; (required values; C, 58.88; H, 6.79).

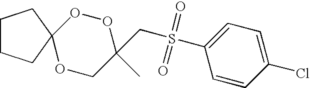

8-Benzenesulfonylmethyl-8-methyl-6,7,10-trioxa-spiro[4.5]decane (8d). This compound was prepared in 96% yield according to General Procedure 2. Mp 138-139° C. ¹H NMR (400 MHz, CDCl₃) δ_{H} 7.90 (d, J=8.40, 2H, aromatic), 7.55 (d, J=8.60, 2H, aromatic), 3.99 (d, J=12.08, 1H, O—CH), 3.90 (d, J=14.16, 1H, O—CH), 3.71 (t, J=13.96, 2H, —CH₂), 1.77 (m, 4H, cyclopentyl moiety), 1.59 (m, 4H, cyclopentyl moiety); ¹³C NMR (100 MHz, CDCl₃) δ_{c} 139.75, 129.85, 114.93, 67.58, 58.77, 37.38, 32.25, 24.98, 23.53 and 20.26. MS (ES+) m/z 369.1 [M+Na]⁺ (100), 385.1 [M+K]⁺ (12), 715.2 [2M+Na]⁺ (20); HRMS m/z calcd for C₁₅H₁₉O₅SClNa [M⁺+Na] 369.0526 found, 369.0539; Elemental analysis C, 52.40; H, 5.45; (required values; C, 51.95; H, 5.52).

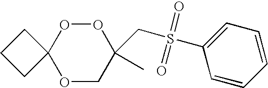

7-Benzenesulfonylmethyl-7-methyl-5,6,9-trioxa-spiro[3.5]nonane (8e). This compound was prepared in 91% yield according to General Procedure 2. Mp 96-97° C. ¹H NMR (400 MHz, CDCl₃) δ_{H} 7.96 (d, J=8.15, 2H, aromatic), 7.66 (t, J=7.44, 1H, aromatic), 7.57 (t, J=7.76, 2H, aromatic), 4.06 (bs, 2H, —CH₂), 3.64 (d, J=11.92, 1H, O—CH), 3.57 (d, J=15.24, 1H, O—CH), 2.24 (m, 4H, cyclobutyl moiety), 1.76 (m, 2H, cyclobutyl moiety), 1.44 (s, 3H, —CH₃); ¹³C NMR (100 MHz, CDCl₃) δ_{c} 141.42, 134.07, 129.61, 128.19, 108.80, 104.98, 66.26, 31.00, 20.20 and 11.86; MS (ES+) m/z 321.1 [M+Na]⁺ (100), 337.3 [M+K]⁺ (5); HRMS m/z calcd for C₁₄H₁₈O₅SNa [M+Na]⁺ 321.0773 found, 321.0771; Elemental analysis C, 56.50; H, 6.09; (required values; C, 56.36; H, 6.08).

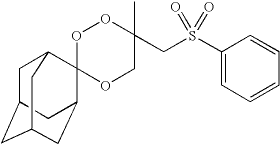

Adamantyl sulfone trioxane (8f); This compound was prepared in 56% yield according to General Procedure 2. Mp 126-127° C. ¹H NMR (400 MHz, CDCl₃) OH 7.96 (d, J=7.12 2H, aromatic signal), 7.64 (t, J=7.44, 1H, aromatic signal), 7.55 (t, J=7.12, 2H, aromatic), 3.92 (bd, J=11.28 2H, —CH₂), 3.76 (d, J=12.08, 1H, O—CH trioxane moiety), 3.66 (bd, J=12.08, 1H, O—CH), 2.05-1.53 (m, 14H, adamantane moiety), 1.46 (s, 3H, —CH₃); ¹³C NMR (100 MHz, CDCl₃) δ_{c} 141.48, 133.91, 129.45, 128.30, 104.87, 64.47, 59.24, 37.40, 33.75, 33.57, 33.21, 27.41 and 20.39. MS (ES+) m/z 401.1 [M+Na]⁺ (100); HRMS m/z calcd for C₂₀H₂₆O₅SNa [M+Na]⁺ 401.1399 found, 401.1379 Elemental analysis C, 63.45; H, 6.95; (required values; C, 63.47; H, 6.92).

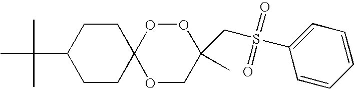

3-Benzenesulfonylmethyl-9-tert-butyl-3-methyl-1,2,5-trioxa-spiro[5.5]undecane (8g). This compound was prepared in 92% yield according to General Procedure 2. Mp 154° C. ¹H NMR (400 MHz, CDCl₃) δ_{H} (d, J=8.56, 2H, aromatic), 7.53 (d, J=8.56, 2H, aromatic), 3.81 (bs, 2H, —CH₂—SO₂), 3.73 (bs, 2H, —OCH₂), 1.46 (s, 3H, —CH₃), 1.27 (bm, 4H, cyclohexyl moiety), 0.90 (m, 1H, cyclohexyl moiety), 0.82 (bm, 4H, cyclohexyl moiety); ¹³C NMR (100 MHz, CDCl₃) δ_{c} 139.73, 135.01, 130.12, 129.40, 103.01, 65.48, 47.47, 32.62, 31.98, 27.89, 23.51, 20.26 and 14.52 MS (ES+) m/z 439.13 [M+Na]⁺ (100), 455.11 [M+K]⁺ (10); HRMS m/z calcd for C₂₀H₂₉O₅SClNa [M⁺+Na] 439.1322 found, 439.1344; Elemental analysis C, 57.53; H, 6.89; (required values; C, 57.60; H, 7.00).

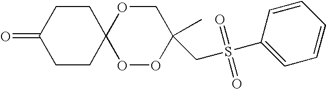

3-Benzenesulfonylmethyl-3-methyl-1,2,5-trioxa-spiro[5.5]undecan-9-one (8h). This compound was prepared in 82% yield according to General Procedure 2. Mp 96° C. 1H NMR (400 MHz, CDCl3) δ_{H} 7.98 (d, J=7.15, 2H, aromatic signal), 7.67 (t, J=7.47, 1H, aromatic), 7.58 (t, J=7.16, 2H, aromatic) 4.12 (q, J=7.15, 2H, —CH2), 3.82 (dd, J=12.24, 0.8, 2H, O—CH), 2.38 (bm, 4H), 1.25 (bm, 4H), 0.89 (t, J=7.00, 3H, —CH₃); ¹³C NMR (100 MHz, CDCl₃) δ_{c} 209.21 (C═O), 140.97, 133.82, 133.70, 129.43, 128.28, 127.89, 65.28, 36.39, 36.17, 29.71 and 20.15. MS (ES+) m/z 363.19 [M+Na]⁺ (100), 379.17 [M+K]⁺ (49), 395.23 [M+Na+CH₃OH]⁺ (55), 411.21 [M+K+CH₃OH]⁺ (25); HRMS m/z calcd for C₁₆H₂₀O₆SNa [M+Na]⁺ 363.0878 found, 363.0867; Elemental analysi

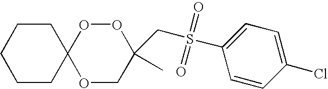

3-(4-Chloro-benzenesulfonylmethyl)-3-methyl-1,2,5-trioxa-spiro[5.5]undecane (8i). This compound was prepared in 83% yield according to General Procedure 2. Mp 86-87° C.; ¹H NMR (400 MHz, CDCl₃) δ_{H} 7.89 (d, J=8.74, 2H, aromatic), 7.53 (t, J=8.74, 2H aromatic), 3.87 (d, J=11.13, 1H, O—CH), 3.79 (d, J=11.92, 1H, O—CH), 3.73 (bs, 2H, —CH₂), 1.71 (bm, 4H, -cyclohexyl moiety), 1.46 (bm, 6H, cyclohexyl moiety), 1.26 (s, 3H, —CH₃); ¹³C NMR (100 MHz, CDCl₃) δ_{c} 141.59, 140.71, 130.97, 130.55, 103.54, 65.46, 59.48, 30.25, 25.88, 22.70 and 20.45.MS (ES+) m/z 383.1 [M+Na]⁺ (100); HRMS m/z calcd for C₁₆H₂₁O₅ClNa [M+Na]⁺ 383.0696 found, 383.0681; Elemental analysis C, 51.83; H, 5.75; (required values; C, 53.26; H, 5.87).

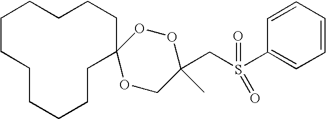

3-Benzenesulfonylmethyl-3-methyl-1,2,5-trioxa-spiro[5.11]heptadecane (8k). This compound was prepared in 93% yield according to general procedure 2. Mp 123-124° C. ¹H NMR (400 MHz, CDCl₃) ⁶H 7.96 (d, J=7.28, 2H, aromatic), 7.65 (t, J=7.44, 2H, aromatic), 3.94 (bd, J=11.76, 2H, —CH₂SO₂—), 3.74 (d, J=12.08, 1H, —O—CH), 3.62 (bd, J=13.32, 1H, —O—CH), 1.55 (s, 3H, —CH₃), 1.32 (bm, 22H, cyclododecane moiety); ¹³C NMR (100 MHz, CDCl₃) δ_{c} 141.49, 133.98, 129.54, 128.23, 106.97, 65.25, 26.36, 22.68, 22.14, 20.44, 19.65 and 18.97; MS (ES+) m/z 433.2 [M+Na]⁺ (100), 449.2 [M+K]⁺ (5); HRMS m/z calcd for C₂₂H₃₄O₅Na [M⁺+Na] 433.2025 found, 433.2032; Elemental analysis C, 65.47; H, 8.72; (required values; C, 64.36; H, 8.35; C, 57.00; H, 5.82; (required values; C, 56.76; H, 5.92).

Adamantyl Trioxane (8m)

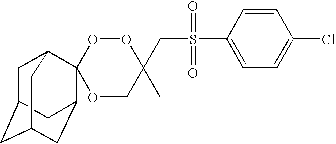

This compound was prepared using the general procedure 80%.

¹H NMR (400 MHz, CDCl₃) δ_{H} 1.45 (bs, 3H, CH₃), 1.50-1.85 (m, 4H, CH₂-adamantyl moiety), 1.90-2.10 (m, 9H, damantly moiety), 3.8 (bs, 4H, SO₂CH₂/CH₂O), 7.55 (d, 2H, Ar), 7.95 (d, 2H, Ar); ¹³CNMR (400 MHz, CDCl₃), 140.61, 139.77, 130.00, 129.62, 104.83, 64.60, 60.74, 59.12, 37.30, 36.42, 33.66, 33.46, 33.10, 28.84, 27.46, 27.32, 20.29, 14.56 MS (ES+) m/z 412.9275 [M+Na]⁺ (100) 435/437, [2M+Na]⁺ (8%) 847/850 HRMS m/z calculated for C₂₀H₂₅O₅NaSCl 435.1009, found, 435.0988

General Procedure 3 Pummerer Reaction

Synthesis of 3-Methyl-1,2,5-trioxa-spiro[5.11]heptadecane-3-carbaldehyde.

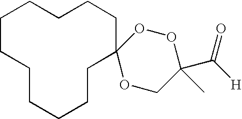

To a solution of the sulphoxide analogue of 4k (1.00 g, 2.33 mmol) at 0° C. in CH₃CN (7 ml), 2,6-lutidine (0.60 ml, 5.12 mmol) and TFAA (0.65 ml, 4.66 mmol), in CH₃CN (5 ml) were added. The mixture was stirred at rt for 3 h, treated with saturated aqueous NaHCO₃ (13 ml) and extracted with AcOEt (3×7 ml). The organic layer was dried (Na₂SO₄) and the solvent was removed under reduced pressure to give the aldehyde as an oily residue in 68% yield (0.53 g). Mp 88-89° C. ¹H NMR (400 MHz, CDCl₃) δ_{H} 9.88 (d, J=2.13, 1H, —CO—H), 4.08 (d, J=11.68, 1H, O—CH), 3.76 (dd, J=11.68, 2.16, 1H, —O—CH), 1.39 (m, 22H, cyclododecanone moiety), 1.08 (s, 3H, —CH₃); ¹³C NMR (100 MHz, CDCl₃) δ_{c} 203.23 (C═O), 106.84, 84.44, 62.21, 26.44, 22.70, 19.91, 18.99 and 16.82; MS (ES+) m/z 307.2 [M+Na]⁺ (25), 339.2 [M+Na+CH₃OH]⁺ (100); HRMS m/z calcd for C₁₆H₂₈O₄Na [M+Na]+307.1885 found 307.1879; Elemental analysis C, 67.94; H, 9.95; (required values; C, 67.57; H, 9.92).

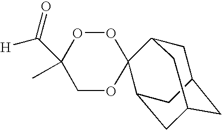

Adamantyl trioxane aldehyde. This compound was prepared in 76% yield according to the General procedure 3. ¹H NMR (400 MHz, CDCl₃) δ_{H} 9.90 (d, J=2.23, 1H, —CO—H), 4.10 (d, J=11.76, 1H, —O—CH), 3.79 (dd, J=11.76, 2.23, 1H, —O—CH), 1.81 (m, 14H, adamantine moiety), 1.08 (s, 3H, —CH₃); ¹³C NMR (100 MHz, CDCl₃) δ_{c} 203.45 (C═O), 104.89, 84.50, 61.58, 37.45, 36.71, 33.62, 27.43 and 16.74; MS (ES+) m/z 275.1 [M+Na]⁺ (32), 291.2 [M+K]⁺ (12), 307.2 [M+Na+CH₃OH]⁺ (100); HRMS m/z calcd for C₁₄H₂₀O₄Na [M+Na]⁺ 275.1259 found 275.1242; Elemental analysis C, 64.22; H, 7.45; (required values; C, 66.65; H, 7.99).

General Procedure 4—Wittig Reactions.
Synthesis of 3-Methyl-3-styryl-1,2,5-trioxa-spiro[5.11]heptadecane.

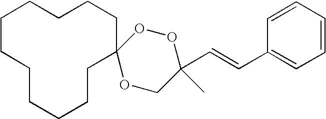

To a stirred suspension of benzyltriphenylphosphonium bromide (0.51 g, 1.18 mmol), in THF (2 ml) was added NHMDS (1.18 ml, 1.18 mmol, 1M solution in THF) via syringe. The reaction mixture was stirred at room temperature for 15 mins, and then a solution of 3-Methyl-1,2,5-trioxa-spiro[5.11]heptadecane-3-carbaldehyde (0.21 g, 0.738 mmol) in THF (2 ml) was added. After being stirred for a further 1 h, the reaction was quenched with saturated aq. NaHCO₃, extracted with ether, washed with brine, dried (Na₂SO₄) and concentrated in vacuo. The product was purified by flash chromatography to give the desired compound as a white solid in 75% yield. Mp 86-87° C. ¹H NMR (400 MHz, CDCl₃) δ_{H} 7.32 (m, 5H, aromatic), 7.11 (d, J=12.67, 1H, trans olefin —C═CH), 6.72 (d, J=12.71, trans olefin), 3.90 (bs, 1H, —OCH), 3.61 (bs, 1H, —O—CH), 1.28 (m, 22H, cyclodeodecane moiety), 1.23 (s, 3H, —CH₃); ¹³C NMR

MHz, CDCl₃) 134.17, 129.17, 128.90, 128.29, 126.92, 126.84, 106.55, 79.41, 60.71, 40.70, 31.94, 26.36, 22.99 and 21.34. MS (ES+) m/z 381.3 [M+Na]⁺ (98), 413.3 [M+CH₃OH+Na]⁺ (62); HRMS m/z calcd for C₂₃H₃₄O₃Na [M+Na]+381.2406 found, 381.2419; Elemental analysis C, 78.26; H, 9.48; (required values; C, 77.05; H, 9.56).

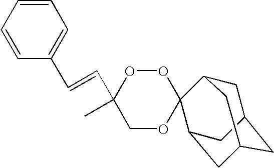

Adamantyl trioxane olefin. This compound was prepared in 71% yield according to General procedure 4. ¹H NMR (400 MHz, CDCl₃) δ_{H} 7.30 (m, 5H, aromatic), 6.70 (d, J=12.8, trans olefin —C═CH), 3.90 (bs, 1H, —O—CH), 3.71 (bs, 1H, —O—CH), 1.75 (m, 14H, adamantine moiety), 1.26 (s, 3H, CH₃); ¹³C NMR (100 MHz, CDCl₃) δ_{c} 133.56, 128.89, 128.58, 127.92, 126.65, 124.92, 110.45, 77.34, 76.70, 37.94, 37.26, 33.47, 27.24 and 21.06; MS (ES+) m/z 349.2 [M+Na]⁺ (100), 365.2 (M+K]⁺ (20); HRMS m/z calcd for C₂₁H₂₆O₃Na [M+Na]⁺ 349.1780 found, 349.1763.

General Procedure 5—Reductive Amination
Synthesis of Adamantyl Trioxane N-Phenyl Substituted Piperazine

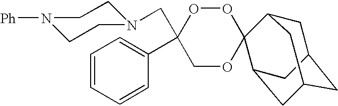

Adamantyl trioxane aldehyde (0.106 g, 0.423 mmol) and N-phenyl piperazine (0.07 ml, 0.465 mmol) were mixed together in 1,2-dichloroethane (14 ml) and then treated with sodium triacetoxyborohydride (0.13 g, 0.599 mmol) and AcOH (0.03 g, 0.423 mmol). The mixture was stirred at rt for 24 h until the reactants were consumed as determined by TLC. The reaction was quenched by adding 1 N NaOH, and the product was extracted with ether. The ether extract was washed with brine and dried (MgSO₄). The solvent was evaporated and the crude product was subsequently purified by flash column chromatography (10:90 MeOH/DCM), affording the desired compound as a yellow oil (90 mg, 54%); MS (ES+) m/z 399.2 [M+H]⁺ (100), 400.3 [M+2H]⁺ (24); HRMS m/z calcd for C₂₄H₃₅O₃N₂ [M+H]⁺ 399.2648 found 399.2649.

General Procedure 6

To a solution of the aldehyde (1.4 mmol) in 12 ml of CH₂Cl₂ was added Ph₃P═CHCO₂Me (1.5 mmol) at room temperature and stirred at this temperature for 3 hours. The reaction mixture was concentrated and chromatographed on a silica gel to give the desired product.

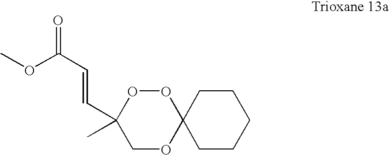

This compound was prepared using the general procedure 6 above in 61%.

¹HNMR (400 MHz, CDCl₃) δ_{H} 1.1-2.25 (m, 13H, CH₃/CH₂-cyclohexyl), 3.8 (s, 5H, CH₂O/CH₃O), 6.15 (d, 1H, CH), 7.18 (bs, 1H, CH), ¹³CNMR (400 MHz, CDCl₃) 166.95, 149.27, 122.16, 102.76, 78.90, 65.18, 52.08, 34.54, 28.77, 25.81, 22.68, 21.65 MS (ES+) m/z 256.2949, [M+Na]⁺ (100) 279, HRMS m/z calculated for C₁₃H₂₀O₅Na 279.1208, found, 279.1201

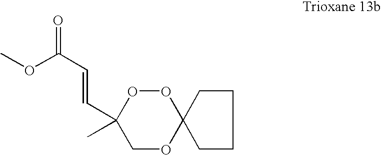

This compound was prepare by the general procedure above in 62%.

¹H NMR (400 MHz, CDCl₃) δ_{H} 1.2 (bs, 3H, CH₃), 1.6-1.9 (m, 10H, CH₂-cyclopentyl), 3.75 (s, 3H, CH₃O), 3.8 (s, 2H, CH₂O), 6.15 (d, 1H, CH), 7.18 (bs, 1H, CH), ¹³CNMR (400 MHz, CDCl₃) 166.95, 149.10, 122.35, 114.71, 78.86, 67.84, 52.11, 37.43, 32.33, 24.82, 23.80, 21.78.

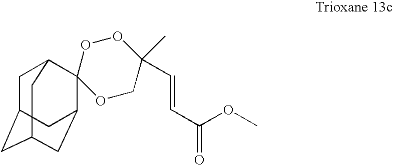

This compound was prepared by the general procedure above in 72%.

¹HNMR (400 MHz, CDCl₃) δ_{H} 1.15 (bs, 3H, CH₃), 1.5-17 (m, 4H, CH₂-adamantly moiety), 1.8 (bs, 4H, CH-adamantyl moiety), 1.85-2.0 (m, 5H, CH₂-adamantly moiety), 3.75 (s, 5H, CH₂O/CH₃O), 6.1 (bs, 1H, CH), 7.15 (bs, 1H, CH), ¹³CNMR (400 MHz, CDCl₃) 167.0, 149.40, 122.17, 104.83, 78.69, 64.79, 60,75,52.08, 47.34, 39.62, 37.47, 33.70, 29.15, 27.60, 27.46, 21.71, MS (ES+) m/z, 308.3695 [M+Na]⁺ (100) 331.2 HRMS m/z calculated for C₁₇H₂₄O₅Na 331.1521, found, 331.1520

Trioxepane Synthesis

General Procedure

A 2-necked 500 ml round bottom flask was charged with a solution of 3-phenyl-3-propen-ol (1 g, 7.5 mmol) and AIBN (77.5 mg, 4.72 mmol) in acetonitrile (115 ml). The reaction vessel was flushed with oxygen for several minutes at 0° C. then stopped and kept under a positive pressure of pure oxygen, with the aid of two big oxygen balloons. The reaction mixture was vigorously stirred and UV irradiated at 0° C. using an externally mounted 100W BLACK-RAY UV lamp at a distance of 5-7 cm, with the simultaneous addition of 4-chlorothiophenol (1250 mg, 8.64 mol) solution in acetonitrile (32 ml) over a period of 30 min. After completion of the addition, the reaction was left to continue stirring at 0° C., for 4-6 hours or until consumption of starting materials (monitored by tlc). The reaction vessel was then cooled to −10° C., flushed with nitrogen and a solution of cyclohexanone (1703 mg, 17.35 mmol) in dichloromethane (32 ml) was added followed by catalytic amount of tosic acid. The mixture was left stirring at −10° C., and allowed to cool slowly to room temperature overnight. The solvent was removed by the rotary evaporator and Column Chromatography on the crude mixture gave an oily product in 72%.

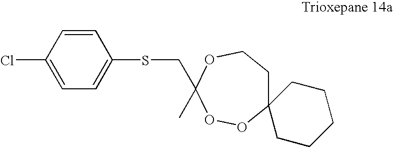

¹HNMR (400 MHz, CDCl₃) δ_{H} 1.25, (s, 3H, CH₃), 1.3-2.52 (m, 12H, CH₂), 3.25 (dd, 1H, SCH₂), 3.5 (d, 1H, SCH₂), 3.75 (t, 2H, OCH₂), 7.25 (d, 2H, Ar), 7.35 (d, 2H, Ar); ¹³CNMR (400 MHz, CDCl₃), 136.23, 132.48, 131.50, 129.34, 106.83, 84.00, 58.91, 44.22, 42.02, 33.61, 25.81, 24.05, 23.43, 23.37, 22.97, 22.78.MS (ES+) m/z 342.8807, [M+Na]^{+ (}100) 365.1/367.1, [2M+Na]+707.2/709.2, HRMS m/z calculated for C₁₇H₂₃NO₃NaSCl 365.0954, found, 365.0940

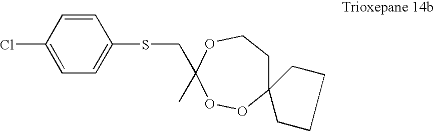

This compound was prepared using the general procedure above in 76% yield as colourless oil. ¹HNMR (400 MHz, CDCl₃) δ_{H} 1.25, (s, 3H, CH₃), 1.6-2.4 (m, 10H, CH₂), 3.2 (dd, 1H, SCH₂), 3.4 (dd, 1H, SCH₂), 3.6-3.8 (m, 2H, OCH₂), 7.25 (dd, 2H, Ar), 7.35 (dd, 2H, Ar); ¹³CNMR (400 MHz, CDCl₃), 136.24, 132.55, 131.28, 129.36, 118.37, 84.23, 60.69, 42.49, 42.23, 34.92, 24.50, 24.43, 24.18, 24.08, 23.96, 23.15 MS (ES+) m/z 328.8541 [M+Na]⁺ (100) 351/353, [2M+Na]⁺ 679/681 HRMS m/z calculated for C₁₆H₂₁NO₃NaSCl 351.0798, found, 351.0786

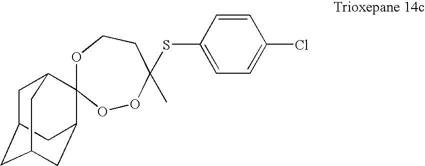

This compound was prepared using the general procedure above in 80% yield as a solid.

¹HNMR (400 MHz, CDCl₃) δ_{H} 1.2 (s, 3H, CH₃), 1.5 (m, 15, CH₂), 1.7 (m, 4H, CH₂), 1.9 (m, 4H, CH₂), 3.15 (d, 1H, SCH₂), 3.45 (d, 1H, SCH₂), 3.6-3.85 (m, 2H, OCH₂), 7.2 (d, 2H, Ar), 7.4 (d, 2H, Ar); ¹³CNMR (400 MHz, CDCl₃), 134.15, 130.14, 128.89, 127.07, 109.64, 106.38, 70.32, 58.15, 56.39, 51.74, 40.13, 40.05, 35.59, 32.65, 32.19, 32.15, 31.73, 31.36 25.36, 21.80, 20.78; MS (ES+) m/z 394. [M+Na]⁺ (100), 417/419, [2M+Na]+811/814, HRMS m/z calculated for C₂₁H₂₇O₃NaSCl 417.1267, found, 417.1280

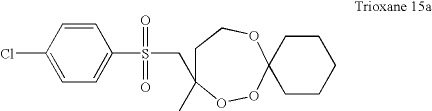

This compound was prepared by the general procedure for the preparation of sulfones above in 81% as white crystal. ¹HNMR (400 MHz, CDCl₃) δ_{H} 1.1 (m, 6H, cyclohexyl), 1.55 (s, 3H, CH₃), 1.9 (m, 4H, cyclohexyl), 2.2 (d, 1H, CH₂), 2.25 (d, 1H, CH₂), 3.35 (1H, SO₂CH₂), 3.7 (d, 1H, SO₂CH₂), 3.8 (t, 2H, CH₂O), 7.55 (d, 2H, Ar), 7.96 (d, 2H, Ar); ¹³CNMR (400 MHz, CDCl₃), 141.19, 140.28, 130.67, 130.19, 107.40, 82.50, 64.83, 62.35, 59.15, 44.25, 42.90, 33.45, 33.13, 26.08, 24.65,23.63, 23.13. MS (ES+) m/z, [M+Na]⁺ (100) [2M+Na]⁺

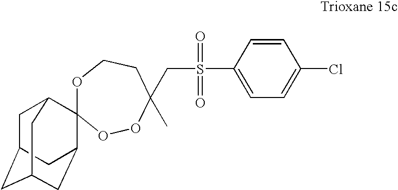

This compound was prepared by the general procedure for the preparation of sulfones in 72% yield as a white crystal. ¹HNMR (400 MHz, CDCl₃) δ_{H} 1.3-1.95 (m, 15, CH₂/adamantyl), 1.55 (s, 3H, CH₃), 3.46 (d, 1H, SO₂CH₂), 3.74 (m, 2H, OCH₂), 3.84 (d, 1H, SO₂CH₂), 7.5 (d, 2H, Ar), 7.95 (d, 2H, Ar); ¹³CNMR (400 MHz, CDCl₃), 140.56, 139.77, 130.40, 129.60, 108.87, 81.73, 61.93, 58.19, 44.21, 37.59, 35.32, 27.63, 23.92 MS (ES+) m/z 426.9541. [M+Na]⁺ (100), 449/451, [2M+Na]⁺ (<5%) 875 HRMS m/z calculated for C₂₁H₂₇NO₄NaS³⁵Cl/C₂₁H₂₇NO₄NaS³⁷Cl 449.1165/451.1136, found, 449.1169/451.1152 respectively.

Preparation of the Methylesters

General Procedure

To a solution of the aldehyde (0.28 g, 1.4 mmol) in 12 ml of CH₂Cl₂ was added Ph₃P═CHCO₂Me (0.5 g, 1.5 mmol) at room temperature and stirred at this temperature for 3 hours. The reaction mixture was concentrated and chromatographed on a silica gel to give the desired product in 62%.

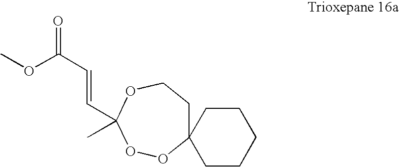

¹HNMR (400 MHz, CDCl₃)6H 1.2 (s, 3H, CH₃), 1.3-1.65 (m, 7H, CH₂), 1.66-2.1 (m, 5H, CH₂), 3.6-3.95 (m, 2H, CH₂O), 3.8 (s, 3H, OCH₃), 5.95 (d, 1H, CH), 7.15 (d, 1H, CH); (400 MHz, CDCl₃), 167.19, 151.88, 14957, 120.27, 107.03, 83.73, 59.02, 52.12, 42.55, 33.54, 32.42, 25.69, 23.38, 22.93 MS (ES+) m/z, 270.3215 [M+Na]⁺ (100) 293, HRMS m/z calculated for C₁₄H₂₂NO₅Na 293.1365, found, 293.1358

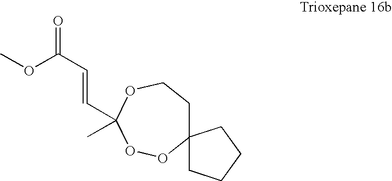

This compound was prepared using the general procedure above in 34%.

¹HNMR (400 MHz, CDCl₃) δ_{H} 1.25 (s, 3H, CH₃), 1.55-1.8 (m, 6H, CH₂), 1.9-2.35 (m. 4H, CH₂), 3.8 (bs, 3H, OCH₃), 3.8 (bs, 2H, CH₂O)5.95 (d, 1H, CH), 7.2 (d, 1H, CH); ¹³CNMR (400 MHz, CDCl₃), 167.14, 151.81, 149.43, 120.11, 118.46, 83.85, 60.85, 52.03, 42.41, 42.18, 35.96, 35.45, 35.40, 25.53, 24.49, 24.34, 24.01, 23.87; MS (ES+) m/z 256.2949. [M+Na]⁺ (100) 279, HRMS m/z calculated for C₁₃H₂₀NO₅Na 279.1208, found, 279.1205

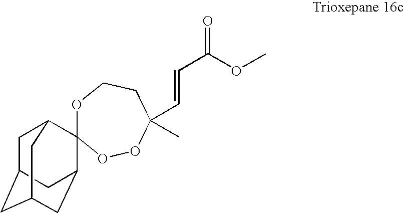

This compound was prepared using the general procedure above in 70% as oil.

¹HNMR (400 MHz, CDCl₃)6H, 1.25 (s, 3H, CH₃), 1.55 (m, 6H, CH₂), 1.8(bs. 4H, CH), 1.95 (m, 4H, CH₂) 3.6-3.95 (m, 2H, CH₂O), 3.8 (s, 2H, OCH₃), 6.0 (d, 1H, CH), 7.25 (d, 1H, CH); ¹³CNMR (400 MHz, CDCl₃), 167.72, 152.62, 120.63, 109.35, 84.07, 59.16, 52.51, 43.10, 38.24, 34.33, 27.97, 26.11; MS (ES+) m/z 322.396. [M+Na]+(100) 345, HRMS m/z calculated for C₁₈H₂₆NO₅Na 345.1678, found, 345.1675

Antimalarial activity. The K1 strain of Plasmodium falciparum was used in this study. This strain is known to be CQ resistant Parasites were maintained in continuous culture using the method of Jensen and Trager (Trager, W; Jenson, J. B. Human Malaria Parasites in Continuous Culture. Science, 1976, 193, 673-675). Cultures were grown in flasks containing human erythrocytes (2-5%) with parasitemia in the range of 1% to 10% suspended in RPMI 1640 medium supplemented with 25 mM HEPES and 32 mM NaHCO₃, and 10% human serum (complete medium). Cultures were gassed with a mixture of 3% O₂, 4% CO₂ and 93% N₂. Antimalarial activity was assessed with an adaption of the 48-h sensitivity assay of Desjardins et al. using [³H]-hypoxanthine incorporation as an assessment of parasite growth (Desjardins, R. E.; Canfield, C. J.; Haynes, J. D.; Chulay, J. D. Quantitative Assessment of Antimalarial activity in vitro by Semi-automated Microdilution Technique. Antimicrob. Agents Chemother., 1979, 16, 710-718). Stock drug solutions were prepared in 100% dimethylsulphoxide (DMSO) and diluted to the appropriate concentration using complete medium. Assays were performed in sterile 96-well microtitre plates, each plate contained 200 μl of parasite culture (2% parasitemia, 0.5% haematocrit) with or without 10 μl drug dilutions. Each drug was tested in triplicate and parasite growth compared to control wells (which consituted 100% parasite growth). After 24-h incubation at 37° C., 0.5 μCi hypoxanthine was added to each well. Cultures were incubated for a further 24 h before they were harvested onto filter-mats, dried for 1 h at 55° C. and counted using a Wallac 1450 Microbeta Trilux Liquid scintillation and luminescence counter. IC₅₀ values were calculated by interpolation of the probit transformation of the log dose-response curve.

1. A 1,2,4-trioxane compound according to structure (A) 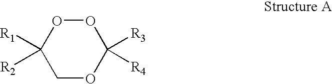
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
or R³ and R⁴ together comprise cycloalkyl,
or an enantiomer, salt, or hydrate thereof.

2. The 1,2,4-trioxane compound according to claim 1 wherein R³ and R⁴ independently or together are adamantyl, cyclopentyl, cyclohexyl, or cyclododecanyl.

3. The 1,2,4-trioxane compound according to claim 1, wherein R¹ is alkyl and R² is aryl.

4. The 1,2,4-trioxane compound according to claim 1, selected from the group consisting of:
8-(4-chloro-phenylsulfanylmethyl)-methyl-6,7,10 trioxa-spiro[4.5]decane;
3-methyl-3-phenylsulfanylmethyl-1,2,5-trioxa-spiro[5.5]undecane;
7-methyl-7-phenylsulfanylmethyl-5,6,9-trioxa-spiro[3.5]nonane, adamantyltrioxane;
9-tert-butyl-3-(4-chloro-phenylsulfanylmethyl)-3-methyl-1,2,5-trioxa-spiro[5.5]undecane;
trioxane ketone (4h), 3-(4-chloro-phenylsulfanylmethyl)-3-methyl-1,2,5-trioxa-spiro[5.5]undecane;
3-methyl-3-phenylsulfanylmethyl-1,2,5-trioxa-spiro[5.11]heptadecane;
3-(4-chloro-phenylsulfanylmethyl)-3-methyl-1,2,5-trioxa-spiro[5.11]heptadecane;
3-methyl-3-(naphthalene-2-ylsulfanylmethyl)-1,2,5-trioxa-spiro[5.11]heptadecane, adamantine trioxane (4m), trioxane (4n);
3-benzenesulfonylmethyl-3-methyl-1, 2, 5-trioxa-spiro[5.5]undecane;
3-(4-chloro-benzenesulfonylmethyl)-3-methyl-1,2,5-trioxa-spiro[5.5]undecane;
7-benzenesulfonylmethyl-7-methyl-5,6,9-trioxa-spiro[3.5]nonane;
adamantyl sulfone trioxane (8f);
3-benzenesulfonylmethyl-9-tert-butyl-3-methyl-1,2,5-trioxa-spiro[5.5]undecane;
8-benzenesulfonylmethyl-9-tert-butyl-3-methyl-1,2,5-trioxa-spiro[5.5]undecane;
8-benzenesulfonylmethyl-8-methyl-6,7,10-trioxa-spiro[4.5]decane;
3-benzenesulfonylmethyl-3-methyl-1,2,5-trioxa-spiro[5.11]heptadecane;
3-benzenesulfonylmethyl-3-methyl-1,2,5-trioxa-spiro[5.5]undecan-9-one;
3-methyl-3-styryl-1,2,5-trioxa-spiro[5.11]heptadecane;
adamantly trioxane olefin;
and an adamantyl trioxane N-phenyl substituted piperazine, or an enantiomer, salt, or hydrate thereof.

5. A pharmaceutical composition comprising a 1,2,4-trioxane compound according to structure (A) 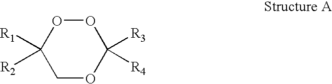
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
or R³ and R⁴ together comprise cycloalkyl,
or an enantiomer, salt, or hydrate thereof,
as an active ingredient in a pharmaceutically acceptable carrier.

6. A method of inhibiting cancer cell proliferation, said method comprising administering to a subject in need thereof an effective amount of a 1,2,4-trioxane compound according to structure (A) 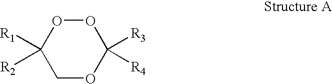
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
or R³ and R⁴ together comprise cycloalkyl,
or an enantiomer, salt, or hydrate thereof, or a compound of structure (B) 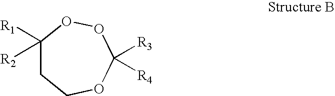
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
wherein R³ and R⁴ may be cyclic ring systems such as adamantyl, cyclopentyl, cyclohexyl and cyclododecanyl, R¹=alkyl and R²=aryl or an enantiomer, salt, or hydrate thereof.

7. A method of treating malaria, said method comprising administering to a subject in need thereof an effective amount of a 1,2,4-trioxane compound according to structure (A) 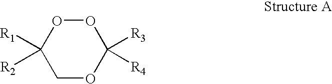
R¹=—CH₂OH, CHO, alkenyl, methyl sulfonyl aryl, methyl sulfinyl, methyl piperazinyl
R²=Aryl, alkyl
R³=Alkyl, cycloalkyl
R⁴=Alkyl, cycloalkyl
or R³ and R⁴ together comprise cycloalkyl,
or an enantiomer, salt, or hydrate thereof, or a compound of structure (B) 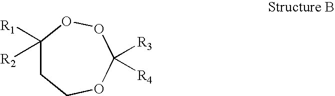
R¹=—CH₂OH, CHO, alkenyl methyl sulfonyl aryl, methyl sulfinyl, methyl piperazinyl
R²=Aryl, alkyl
R³=Alkyl, cycloalkyl
R⁴=Alkyl, cycloalkyl
wherein R³ and R⁴ may be cyclic ring systems such as adamantyl, cyclopentyl, cyclohexyl and cyclododecanyl, R¹=alkyl and R²=aryl or an enantiomer, salt, or hydrate thereof.

8. The method according to claim 7, further comprising co-administration of a second antimalarial compound.

9. The method according to claim 8, wherein said second antimalarial compound is selected from the group consisting of quinoline (amodiaquine), quinoline menthanol (mefloquine), halofantrine, benflumetol and LAPDAP.

10. A method for the synthesis of a 1,2,4-trioxane compound, said method comprising reacting an allylic alcohol and a ketone by a thiol-olefin co-oxygenation (TOCO) reaction.

11. The method of claim 10, wherein said ketone is a cyclic ketone.

12. The method of claim 10, wherein said allylic alcohol is R₁—C(═CH₂)—CH₂—OH and said ketone is R₂—C(═O)—R₃.

13. The method of claim 10, wherein said reacting step comprises:
(a) UV irradiating a mixture comprising said allylic alcohol, AIBN, an optionally substituted thiophenol, O₂, and an aprotic solvent; and
(b) adding to said mixture said ketone and a catalytic amount of tosic acid.

14. The method of claim 9, wherein said 1,2,4-trioxane is a 1,2,4-trioxane sulfide, and further comprising the step of converting said sulfide to a 1,2,4-trioxane sulfone.

15. The method of claim 10, wherein said 1,2,4-trioxane is a 1,2,4-trioxane sulfide, and further comprising the steps of (c) converting said 1,2,4-trioxane sulfide to a 1,2,4-trioxane sulfoxide by sulfoxidation, and (d) converting said 1,2,4-trioxane sulfoxide to a 1,2,4-trioxane aldehyde by a Pummerer reaction.

16. The method of claim 15, further comprising the step of derivatizing said 1,2,4-trioxane aldehyde.

17. The method of claim 16, wherein said derivatizing step is a condensation or nucleophilic substitution reaction.

18. The method of claim 17, wherein said derivatizing step is a Wittig reaction or a reductive amination.

19. A method for generating a 1,2,4-trioxane by a TOCO/condensation reaction, the method comprising:
(a) reacting an allyl alcohol with a phenylthiyl radical to generate a tertiary carbon radical,
(b) reacting the radical to trap oxygen to form a peroxy radical,
(c) abstracting radical hydrogen from thiophenol to produce an α-hydroxyperoxide,
(d) condensing the α-hydroxyperoxide with cyclohexanone to generate a 1,2,4-trioxane.

20. A method for generating a 1,2,4-trioxepane by a TOCO/condensation reaction, the method comprising:
(a) reacting a homoallylic alcohol with a phenylthiyl radical to generate a tertiary carbon radical,
(b) reacting the radical to trap oxygen to form a peroxy radical,
(c) abstracting radical hydrogen from thiophenol to produce an α-hydroxyperoxide,
(d) condensing the α-hydroxyperoxide with cyclohexanone to generate a 1,2,4-trioxepane.
